(19) United States
(12) Patent Application Publication
(10) Pub. No.: US 2022/0031306 A1
(43) Pub. Date: Feb. 3, 2022

(54) SURGICAL CANNULA WITH CLOSURE GUIDES

(71) Applicant: Tarek S. Hassan, Ann Arbor, MI (US)

(72) Inventor: Tarek S. Hassan, Ann Arbor, MI (US)

(21) Appl. No.: 16/941,313

(22) Filed: Jul. 28, 2020

(51) Int. Cl. A61B 17/04; A61B 17/34; A61F 9/007; A61B 17/06

(52) U.S. Cl. CPC A61B 17/0482; A61B 17/3421; A61B 2017/0608; A61B 17/06166; A61F 9/007

(57) ABSTRACT

The distal portion of a tissue-penetrating device includes a structure to guide a closure device associated with closing the tissue layer while the device remains in position, prior to removal of the device. The proximal portion of the device includes a visual and/or tactile indication corresponding to the guide structure. The guide structure may include a plurality of passageways such as slits or gaps to pass a suture, for example. The device may assume the shape of a conventional cylindrical cannula having inner and outer diameters defining a cylindrical wall, in which case the guide structure may include a plurality of passageways in the wall. The cannula may have any gauge associated with any surgical procedure, including procedures involving large wounds or incisions, and microsurgical procedures involving minute punctures, including vitrectomies.

### FIELD OF THE INVENTION

This invention relates generally to invasive surgical procedures, including minimally invasive procedures, and, in particular, to improved closure of incision sites using cannulas and other devices adapted for use in such procedures.

### BACKGROUND OF THE INVENTION

Many surgical procedures now use trocars and cannulas, or other mechanical hardware devices, to access internal body spaces and cavities. For example, a pointed trocar within a hollow, tubular cannula may be used to pierce tissue layers. The trocar is removed, leaving the cannula as a portal for the subsequent placement of instruments, such as graspers, scissors, staplers, and so forth.

There are many trocar/cannula systems, and other instruments that allow entry into body cavities with other devices, used for different medical purposes, including laparoscopic abdominal and microsurgical procedures in a number of surgical sites throughout the body. In each case, when the instruments, cannulas, and/or other entry devices are removed, an open puncture wound remains that often must be closed.

In some cases, wound closure with sutures, glues, or other substances may be time-consuming or problematic. During vitrectomy, for example, particularly done with larger-gauge (i.e., 23-gauge) instruments, the sclerotomy wounds can leak, forcing the surgeon to close them with suture after the cannula is removed. This can be challenging and tedious, particularly when the conjunctiva bleeds or balloons up and obscures the incision and can result in a significantly more hemorrhagic and inflamed surgical site. Ophthalmic surgeons often have to guess where to throw the sutures, and apply pressure to the area for up to several minutes to visualize the wounds that need to be closed. Even then, the surgeon may have to cut down the conjunctiva to close the scleral wound, which then increases the tissue invasiveness of the procedure and requires additional sutures to close conjunctiva. Similarly, suture or glue closure of an incisional access point made by a laparoscopic cannula or other incision dilation/maintenance device can be difficult, after removal of the cannula or other such device, if there is extensive bleeding and/or tissue swelling that obscures visualization of the wound edges.

There is an outstanding need for more efficient wound closure devices, systems and methods.

### SUMMARY OF THE INVENTION

The invention improves upon and expedites surgical procedures by providing a tissue-penetrating device with integral closure guidance. A device according to the invention includes a body having a proximal portion, a distal portion, and a proximal-to-distal passage extending through the body. The distal portion of the body is adapted for temporary placement within, or through, an opening in a tissue layer, thereby enabling a user to access an anatomical space through the passage in the body until the body is removed. As used herein, “tissue layer” should be taken to include the skin and any other tissues or membranes, and “anatomical space” should be taken to include any internal region of a human or animal body, including the head, torso, extremities, glands and organs.

The distal portion of the body includes a guide structure to guide a separate device associated with closing the opening in the tissue layer before the body is removed. The proximal portion of the body may include a lip or rim adapted to remain outside of the opening in the tissue layer when the body is in place. In any case, the proximal portion of the body includes a visual and/or tactile indication corresponding to the guide structure.

The guide structure may include a plurality of slits in the distal portion of the body. As used herein, “slit” should be taken to include slots, notches, channels, gaps, separations, and so forth, of any applicable shape or width, so long as the device is usable in accordance with this specification. For example, the slits may be dimensioned to pass a suture.

In the preferred embodiment, the proximal portion of the body includes a visual and/or tactile indicator corresponding the location of each slit, and the slits may have smooth or sharp edges and rounded or straight-edged corners to minimize interference with the placement of the body within or through an opening in a tissue layer.

At least a portion of the device may assume the shape of a conventional cylindrical cannula having proximal and distal ends and inner and outer diameters defining a cylindrical wall. As with the other embodiments disclosed herein, the distal portion of the cannula includes a guide structure to guide a separate device associated with closing a wound or incision before the cannula is removed, and the proximal end of the cannula includes a visual and/or tactile indicator corresponding to the guide structure.

The guide structure may include a plurality of slits in the distal wall of the cannula, with the proximal end of the cannula including a visual and/or tactile indicator corresponding to the location of each slit. The inner diameter of the cannula may define a smooth bore with a gauge associated with any surgical procedure including procedures involving large wounds or incisions, and microsurgical procedure involving minute punctures. The slits in the distal wall of the cannula may be axially aligned with the bore and on opposing sides thereof. The guide structure may include a pair of visual and/or tactile indicators, each indicator being longitudinally aligned with a respective one of the slits. Multiple pairs of slits and corresponding indicators may also be provided.

A method of performing a surgical procedure in accordance with the invention comprises the step of providing a device with a passage between proximal and distal ends, and wherein the distal end of the device includes a guide structure for closing. The device is temporarily inserted into an opening in a tissue layer, enabling a user to access an anatomical space through the passage in the device. While the device is still in position in the opening in the tissue, a separate closure device is inserted through the tissue using the guide structure. The device is removed, leaving the separate closure device remaining through the tissue, whereby the opening in the tissue is closed using the separate closure device.

The proximal end of the device preferably includes visual and/or tactile indicators corresponding to the guide structure, and the method includes the step of referring to the visual and/or tactile indicators to insert the separate closure device through the tissue. The separate closure device may comprise a suture attached to a suture needle. The suture needle may be a curved or straight suture needle, and the method may include the steps of inserting the needle on one side of the passage and removing the needle on an opposing side of the passage through the closure device.

### BRIEF DESCRIPTION OF THE DRAWINGS

FIG. 1 is a drawing that shows a prior art trocar and cannula inserted through one or more layers of tissue;

FIG. 2 is an oblique vie of a preferred embodiment of the invention;

FIG. 3A is an end view of an embodiment of the invention illustrating the use of indicator dots which may be flat or have associated tactile sensation if elevated, corrugated, or with other such surface alteration;

FIG. 3B is a side view of the device of FIG. 3A;

FIG. 4A is a drawing in partial cross section that illustrates a device according to the invention in use just prior to wound closure, with a suture in this example, as part of a vitrectomy procedure; and

FIG. 4B illustrates a second step of a procedure, wherein the inventive device has been removed, leaving a puncture wound in tissue layer(s) and wherein, in this example, the suture in placed to be tied and have the wound closed.

### DETAILED DESCRIPTION OF THE PREFERRED EMBODIMENTS

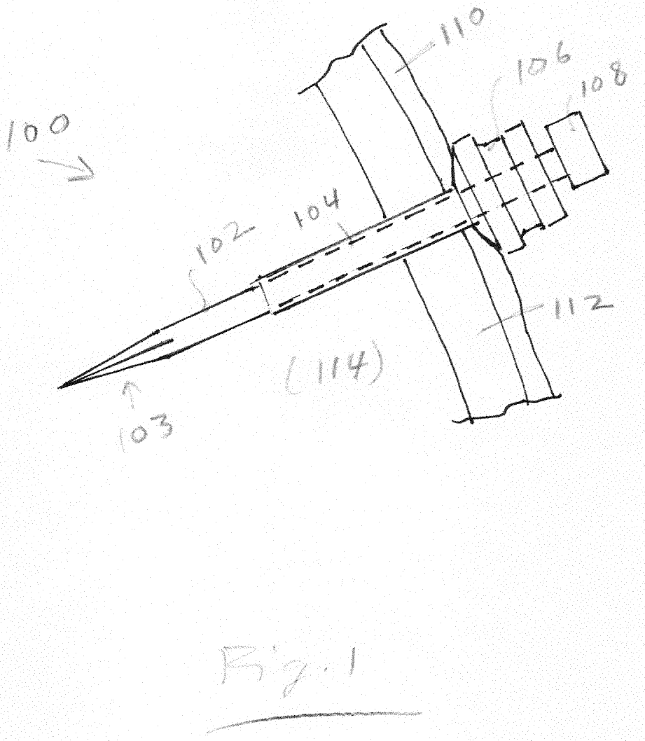

FIG. 1 is drawing that shows generally at 100 a prior-art trocar and cannula inserted through one or more layers of tissue. The trocar 102 has a sharpened tip 103, and is shown within the hollow, tubular sleeve portion 104 of the cannula, which includes portion 106 that remains external, providing a seal. The trocar 102 extends from some inserter portion 108, and tip 103 may include different facets to create a pointed tip.

The trocar/cannula system of FIG. 1 may be used in conjunction with any skin- or tissue-penetrating procedure, including microsurgical procedures such as vitrectomies, in which case layer 112 represents the sclera, 110 may be an outer tissue layer, and 114 represents the vitreous body. A small puncture wound requiring closure is left when the system shown in FIG. 1 is removed following the procedure.

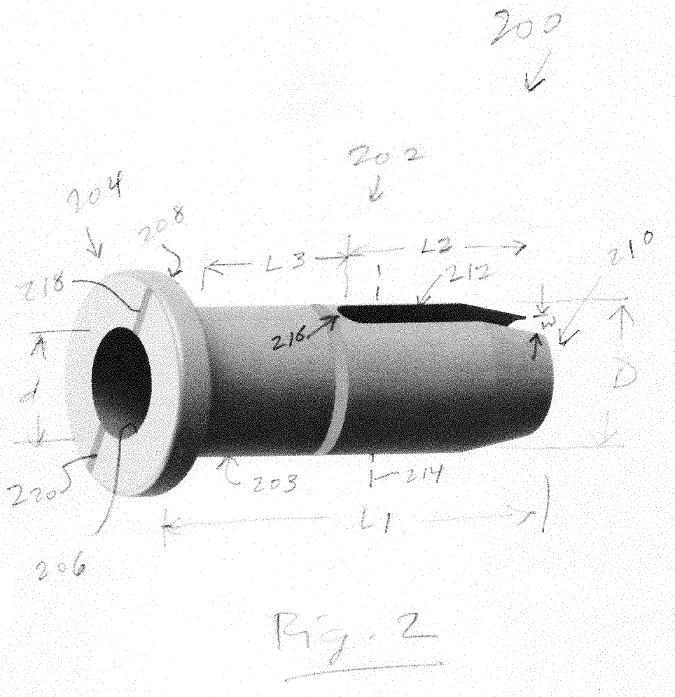

FIG. 2 is an oblique view of an embodiment of the invention. The device comprises a hollow, tubular cannula 200 having a distal, cylindrical sleeve portion 202 with a smooth outer wall 203 having a diameter D. The sleeve portion 202 transitions to a proximal rim portion 204. A cylindrical bore with a smooth, inner wall 206 and diameter d extends axially through the device 200.

The sleeve portion 202 of device 200 defines a length L1 that extends from a lower, distal surface 208 of rim 204 to an end 210. Unique to the invention, the sleeve portion 202 of device 210 includes at least one pair of opposing gaps or slots of width W through the wall of the sleeve portion. Only one of these slots, 212, is visible in FIG. 2, with the understanding that a corresponding slot in the opposing wall of the sleeve creates a gap or slot of width W through the entire device, as shown with broken line 214.

The slots in the device have a length L2 that extend from a proximal end 216 to the distal end 210. The proximal end of each slot 212 is spaced from the lower surface 208 of rim 204 by a distance L3, and preferably includes a rounded beginning 216. The width W may be the same throughout the length L2 of each slot, or may become narrower or wider, as long as the method described below is supported.

The location of each slot is designated with visible indicia on the rim portion 204. For example, the location of slot 214 is designated with marker 218, and the location of an opposing slot, not visible in FIG. 2, is designated with marker 220. In the preferred embodiment, indicia 218, 220 designate the central axis of each slot. Any indicator may be used to form indicia 218, 220, including, without limitation, dots, lines or other shapes made with color, contrast, grooves or raised material.

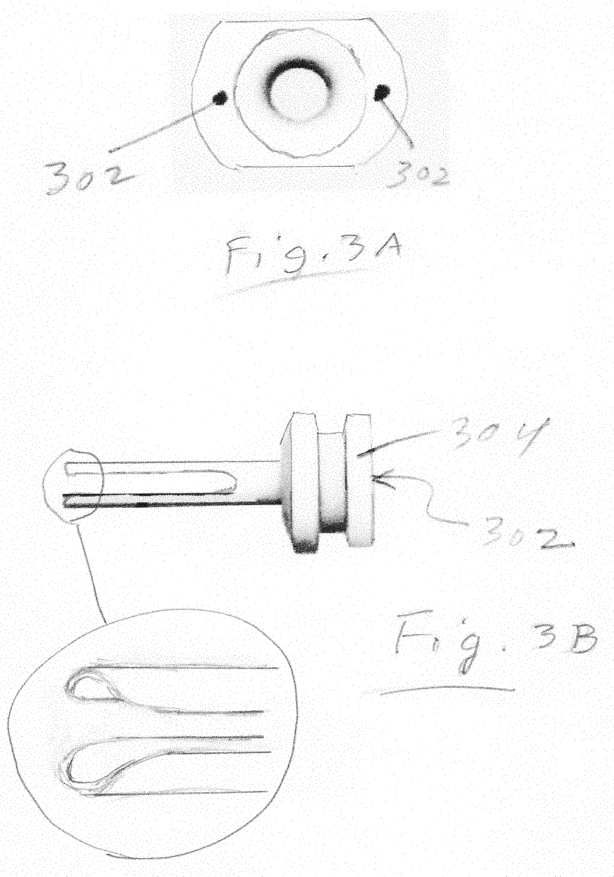

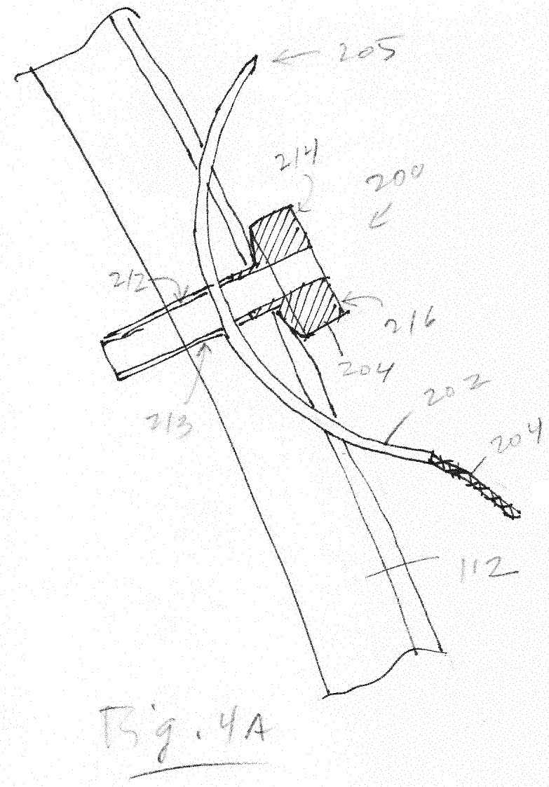

FIG. 3A, for example, shows the use of dot indicators 302. FIG. 3B shows how the opposing gaps in the sleeve form a slot through the device, and also shows a different style of rim portion 304. FIG. 3B also shows how the distal end of the sleeve or shaft portion is beveled, in this example, at 306, 308 where it meets tissue in the region of the slits to avoid a sharp 90 degree angle between the bottom of the cannula and the perpendicular portion of the slit(s).

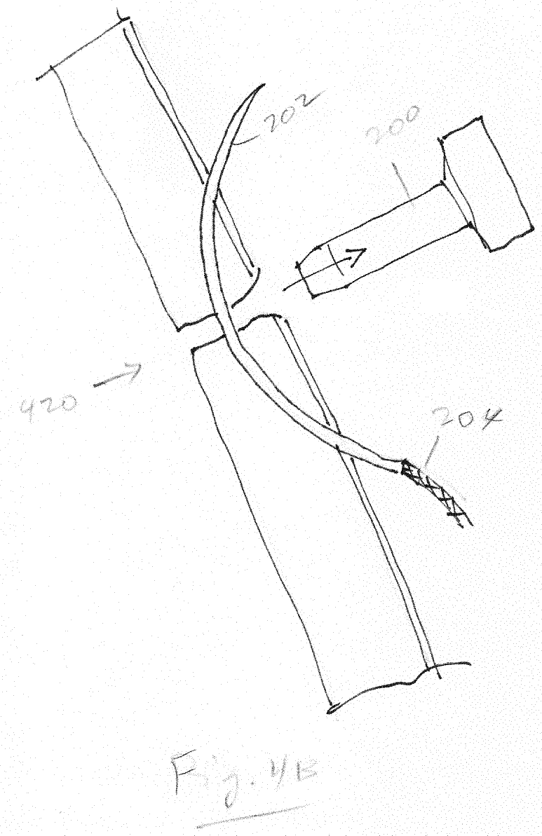

FIG. 4A is a drawing in partial cross section that illustrates a device according to the invention in use just prior to wound closure as part of a vitrectomy procedure. The trocar and all instrumentation used in conjunction with the procedure have been removed, but trocar 200 has been left in place. A curved needle 402, attached to suture material 204, has been inserted through tissue layers, such as sclera 112. Visible indicia 214, 216 on the rim 204 have informed the surgeon precisely where to insert needle 202, and where protruding tip 205 will emerge for grasping.

FIG. 4B illustrates a second step of the procedure, wherein the inventive device 200 has been removed, leaving wound 420 in the tissue layer(s). However, in contrast to existing devices and methods, suture needle 202 and attached suture 204 has already been precisely placed transversely through the center of wound 420, such that by grasping and pulling on needle 202, a suture may be immediately tied to close off the wound without any of the issues associated with prior-art techniques.

The invention is not limited in terms of dimensions or procedure. The dimensions of the device, including diameters D and d, may be selected for any gauge of surgery, including gauges 20, 23, 25 and 27 for retina surgery, up to and including larger gauges for larger incisions made in conjunction with laparoscopic and other procedures involving other parts of the body. Larger gauges would typically use smaller sized slits (L2 and/or W) in the cannula shaft for the suture passes. Length L1 may be selected to adequately penetrate tissue layers associated with a given procedure, and L3 may be chosen to penetrate a particular layer at a particular depth. Nor is the invention limited in terms of the size or style of the outer rim portion 204.

Examples of non-ophthalmic trocar cannula systems applicable to the invention include at least the following, incorporated herein by reference:

https://www.medtronic.com/covidien/en-us/products/trocars-a.ccess.

https://www.medicalexpo.com/medical-manufacturer/laparoscopic-trocar-6591, html

http://www.endoscopysuperstore.com/lap-trocars-and-cannulas.aspx#

The invention is compatible with rigid, semi-rigid and flexible tissue-penetrating device structures, as long as the structure would benefit from the improved closure features and methods made possible by the invention. For example, the invention may be adapted to flexible ports such as the GelPort laparoscopic system offered by Applied Medical. Even with devices that reside within tissue layers as opposed to full tissue penetration, guide structures according to the invention such as gaps, slots, and other forms of separations and passageways may nevertheless be provided on a distal surface of the device to assist with closure guidance.

Further, while the preferred embodiment includes a pair of opposing gaps to create a single passageway slit through the sleeve or shaft of the device, additional, gaps may be provided to provide additional passageways. For example, the wall of the shaft may include four gaps to create two slits at right angles to one another, particularly with larger incisions associated with smaller-gauge procedures. The invention is also not limited in terms of the size or shape of the gaps that create the passageway slits that serve as suture guides as they can be modified depending on the material and method used for closure. With two transverse suture guides, four dots or visual and/or tactile indicators would be provided, and so on. The inventive device may be constructed from any suitable material(s), including polyimide, biocompatible plastics and metals such as stainless steel. The device may be disposable or reusable, and closure materials other than sutures may be accommodated, including clips, staples, and so forth.

1. A surgical device with closure guidance, comprising:
a body having a proximal portion, a distal portion, and a proximal-to-distal passage extending through the body;
wherein the distal portion of the body is adapted for temporary placement within or through an opening in a tissue layer, thereby enabling a user to access an anatomical space through the passage in the body until the body is removed; and
wherein the proximal portion of the body includes a guide structure to guide a separate device associated with closing the opening in the tissue layer before the body is removed.

2. The device of claim 1, wherein the body is a surgical cannula.

3. The device of claim 1, wherein the body is rigid or flexible.

4. The device of claim 1, wherein the proximal portion of the body includes a lip or rim adapted to remain outside of the opening in the tissue layer when the body is in place.

5. The of claim 1, wherein the guide structure includes a visual or tactile indicator on the proximal portion of the body.

6. The device of claim 1, wherein the guide structure includes a plurality of slits in the distal portion of the body.

7. The device of claim 1, wherein:
the guide structure includes a plurality of slits the distal portion of the body; and
the slits are dimensioned to pass a suture or other closure device through the slits before the body is removed.

8. The device of claim 1, wherein:
the guide structure includes a plurality of slits in the distal portion of the body; and
the proximal portion of the body includes a visual or tactile indicator corresponding the location of each slit.

9. The device of claim 1, wherein:
the guide structure includes a plurality of slits in the distal portion of the body; and
the slits have edges and corner structures shaped to minimize interference with the placement of the body within or through an opening in a tissue layer.

10. A surgical device with closure guidance, comprising:
a cylindrical cannula with proximal and distal ends and inner and outer diameters defining a cylindrical wall;
wherein the distal end of the cannula is adapted for temporary placement within or through a wound or incision in a tissue layer enabling a user to access an anatomical space through the cannula until the cannula is removed;
wherein the distal portion of the cannula includes a guide structure adapted to guide a separate device associated with closing the wound or incision in the tissue layer before the cannula is removed; and
wherein the proximal end of the cannula includes a visual or tactile indicator enabling a user to determine the location or orientation of the guide structure while the cannula is in place.

11. The device of claim 10, wherein the cannula is rigid or flexible.

12. The device of claim 10, wherein the proximal end of the cannula includes a lip or rim adapted to remain outside of the incision or wound when the cannula is in place; and
the visual or tactile indicator is on the lip or rim.

13. The device of claim 10, wherein the guide structure includes a plurality of slits in the distal wall of the cannula; and
the proximal end of the cannula includes a visual or tactile indicator corresponding to the location or orientation of each slit.

14. The device of claim 13, wherein the slits are dimensioned to receive a suture or other closure material before the cannula is removed.

15. The device of claim 14, wherein:
the inner diameter defines a smooth bore with a gauge;
the slits are axially aligned with the bore and on opposing sides of the distal wall in the cannula; and
the guide structure includes a pair of visual or tactile indicators, each indicator being longitudinally aligned with a respective one of the slits.

16. The device of claim 15, wherein the slits are physically configured to minimize interference with the placement of the cannula within or through the opening in a tissue layer.

17. The device of claim 15, wherein the gauge is variable in size for a range of procedures including vitrectomies and other microsurgical procedures.

18. A method of performing a surgical procedure, comprising the steps of:
providing the device of claim 1;
temporarily inserting the device into an opening in a tissue layer enabling a user to access an anatomical space through the passage in the device;
while the device is still in position in the opening in the tissue, inserting a separate closure device through the tissue using the guide structure;
removing the device with the separate closure device remaining through the tissue; and
closing the opening in the tissue using the separate closure device.

19. The method of claim 18, wherein the proximal end of the device includes visual indicators corresponding to the guide structure; and
the method includes the step of referring to the visual indicators to insert the separate closure device through the tissue.

20. The method of claim 18, wherein the separate closure device is a suture attached to a suture needle.

21. The method of claim 20, wherein the suture needle is a curved suture needle, and the method includes the steps of inserting the needle on one side of the passage, through the guide structure, and removing the needle on an opposing side of the passage.

22. The method of claim 18, wherein the anatomical space includes an intraocular space.

23. The method of claim 18, wherein the anatomical space includes an abdominal space.

24. The method of claim 18, wherein the tissue layer includes a layer of skin.
